(19) United States
(12) Patent Application Publication
(10) Pub. No.: US 2002/0076701 A1
(43) Pub. Date: Jun. 20, 2002

(54) DNA detection device

(76) Inventors: Keiko Neriishi, Kanagawa (JP); Kenji Takahashi, Kanagawa (JP); Chiyuki Umemoto, Kanagawa (JP); Nobuhiko Ogura, Kanagawa (JP)

Correspondence Address: BIRCH STEWART KOLASCH & BIRCH, PO BOX 747, FALLS CHURCH, VA, 22040-0747, US

(21) Appl. No.: 09/749,791

(22) Filed: Dec. 28, 2000

(30) Foreign Application Priority Data
Dec. 28, 1999 (JP) 11-372976

(51) Int. Cl. C12Q 1/68; C12M 1/34

(52) U.S. Cl. 435/6; 435/287.2

(57) ABSTRACT

A process for detecting a complementary DNA fragment is performed utilizing a DNA micro-array and a radiation image storage panel containing a stimulable phosphor in local areas.

### FIELD OF THE INVENTION

The present invention relates to a process for detecting a complementary DNA fragment method utilizing a DNA micro-array and a radiation image storage panel.

### BACKGROUND OF THE INVENTION

Recently, a DNA micro-array is widely utilized in gene analyzing technology. The DNA micro-array comprises a support (i.e., micro-chip) of an extremely small area (such as approx. 1 mm² or less) on which a group of nucleotide derivatives or their analogues (probes, e.g., DNA fragments, synthesized oligonucleotides or polynucleotide, PNA) are fixed. On one DNA micro-array, various kinds of nucleotide probes are fixed separately from each other.

In gene analyzing technology, detection of DNA fragments complementary to oligonucleotide probes whose base sequence is already known is very important.

The conventional procedure for detecting DNA fragments complementary to oligonucleotide probes are conducted in the following steps:

bringing single-stranded sample DNA fragments having a specific label (e.g., fluorescent label or radioactive label) in an aqueous solution into contact with a DNA micro-array having at least two defined areas in each of which a group of nucleotide derivatives and analogues thereof are fixed under such condition that a group of nucleotide derivatives and analogues thereof fixed in one area differs from a group of nucleotide derivatives and analogues thereof fixed in another area, so that DNA fragments complementary to a group of nucleotide derivatives and analogues thereof are fixed by hybridization to the area in which the group is fixed;

removing unfixed sample DNA fragments from the DNA micro-array; and

detecting the labeled DNA fragments fixed onto the DNA micro-array by hybridization utilizing an appropriate detection procedure.

If a fluorescent label is employed, fluorometry is performed, while a radioactive label is employed, autoradiography is utilized.

The autoradiography utilizing a combination of a radiographic film and a radiographic intensifying screen is favorably employable as the detection procedure. However, since the amount of DNA fragments to be utilized in the detection is extremely small, the autoradiography sometimes shows unsatisfactory sensitivity.

Recently, a radiation image storing and reproducing method utilizing a radiation image storage panel (which is also named “stimulable phosphor sheet” has been widely employed in place of the conventional autoradiography, because the sensitivity provided by the radiation image storage panel is relatively high, as compared with the conventional autoradiographic system.

The use of the autoradiographic procedure utilizing the radiation image storage panel is already known. See Human Molecular Genetics, 1999, Vol. 8, No. 9, 1715-1722.

According to the studies performed by the present inventors, however, the high sensitivity of the radiation image storage panel sometimes shows analytical errors which are caused by the fact that the high sensitive radiation image storage panel absorbs not only the radiation energy emitted by the target DNA fragments (that is, the complementary DNA fragments but also radiation energy emitted by the non-target DNA fragments (that is, non-complementary DNA fragments) which are inadvertently fixed to the DNA micro-array not by hybridization.

### SUMMARY OF THE INVENTION

The present invention provides an improved method for detecting complementary DNA fragments utilizing a combination of the conventional DNA micro-array and the conventional radiation image storage panel, which is almost free from noises caused by the inadvertently fixed non-complementary DNA fragments.

The invention resides in a process for detecting a complementary DNA fragment which comprises the steps of:

bringing single-stranded sample DNA fragments having a radioactive label in a liquid phase into contact with a DNA micro-array having at least two defined areas in each of which a group of nucleotide derivatives and analogues thereof are fixed under such condition that a group of nucleotide derivatives and analogues thereof fixed in one area differs from a group of nucleotide derivatives and analogues thereof fixed in another area, so that DNA fragments complementary to a group of nucleotide derivatives and analogues thereof are fixed by hybridization to the area in which the group is fixed;

removing unfixed sample DNA fragments from the DNA micro-array;

keeping the DNA micro-array in contact with a radiation image storage panel containing a stimulable phosphor in areas corresponding to the areas on which groups of nucleotide derivatives or analogues thereof are fixed, so that the corresponding areas of the stimlable phosphor sheet can absorb and store radiation energy of the radioactive label coming from the fixed DNA fragments through the openings;

irradiating the radiation image storage panel with a stimulating light, so that the image storage panel releases a stimulated emission from the area in which the radiation energy is stored;

detecting the stimulated emission photoelectrically to obtain a series of electric signals; and

processing the electric signals to locate the area in which the complementary DNA fragments are fixed.

In the process of the invention, the radiation image storage panel is irradiated with a stimulating light preferably after it is separated from the DNA micro-array.

The invention also resides in a kit for detecting complementary DNA fragments comprising a DNA micro-array having at least two defined areas in each of which a group of nucleotide derivatives and analogues thereof are fixed under such condition that a group of nucleotide derivatives and analogues thereof fixed in one area differs from a group of nucleotide derivatives and analogues thereof fixed in another area, and a radiation image storage panel containing a stimulable phosphor in areas corresponding to the areas on which groups of nucleotide derivatives or analogues thereof are fixed.

The invention further resides in a composite structure comprising a DNA micro-array having at least two defined areas in each of which a group of nucleotide derivatives and analogues thereof are fixed under such condition that a group of nucleotide derivatives and analogues thereof fixed in one area differs from a group of nucleotide derivatives and analogues thereof fixed in another area, and a radiation image storage panel containing a stimulable phosphor in areas corresponding to the areas on which groups of nucleotide derivatives or analogues thereof are fixed, overlaid in order, the radiation image storage panel be positioned in relation to the DNA micro-array in such condition that the areas containing stimulable phosphor of the radiation image storage panel face the areas of the micro-array in which groups of nucleotide derivatives and analogues thereof are fixed.

### BRIEF DESCRIPTION OF DRAWINGS

FIG. 1 illustrates a composite structure composed of a DNA micro-array and a radiation image storage panel according the invention under the separated condition.

FIG. 2 illustrates a composite structure composed of a DNA micro-array and a radiation image storage panel overlaid in order, for subjecting it to autoradiography.

FIG. 3 illustrates a radiation image storage panel of the invention.

FIG. 4 schematically illustrates a procedure for reproducing a radiation image stored in the radiation image storage panel.

### DETAILED DESCRIPTION OF THE INVENTION

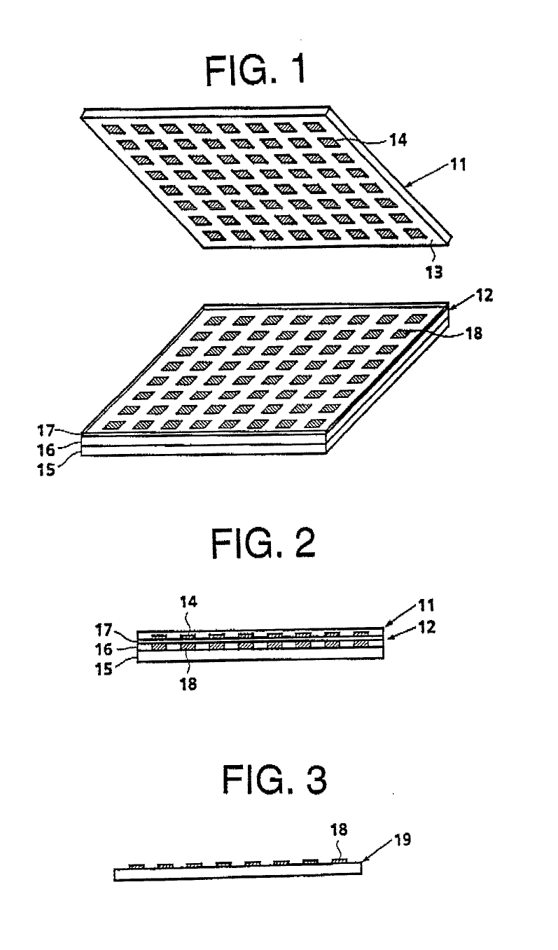

As is shown in FIG. 1 and FIG. 2, the process of the invention utilizes a DNA micro-array 11 and a radiation image storage panel 12.

The DNA micro-array 11 is composed of a support 13 and many micro-chips 14 arranged on the support 13. On each micro-chip 14 are fixed a group of probe compounds such as oligonucleotides. Generally, a group of oligonucleotides fixed in one micro-chip have the essentially same base sequence.

The radiation image storage panel 12 has a support 15 and a stimulable phosphor layer 16 having areas 18 in the positions corresponding to the micro-chips 14 of the DNA micro-array 11, so as to efficiently absorb radiation energy coating from the DNA fragment equipped with a radioactive label. The stimulable phosphor layer 16 is covered with a protective film 17.

The area 18 in the stimulable phosphor layer 16 preferably has a shape and a size corresponding to the micro-chip 14 on which a group of nucleotide probes are fixed.

The area other than the areas containing the stimulable phosphor do not absorb the radiation energy from the area other than the micro-chips 14.

The area other than the areas containing the stimulable phosphor may be covered or enclosed with a non-radiation transmitting material such as metal (e.g., stainless steel, aluminum, copper, or brass), ceramic material (e.g., aluminum oxide, magnesium oxide, silicon nitride, carbon), or polymer material (e.g., polyethylene terephthalate, polyethylene naphthalate, polyurethane, acrylic resin). Otherwise, the areas containing stimulable phosphor can be embedded in the support sheet which is made of non-radiation transmitting material.

In the invention, a thin spacer sheet having openings in the positions corresponding to the micro-chips 14 of the DNA micro-array 11 and the stimulable phosphor-containing areas of the stimulable phosphor sheet, so as to limit the transmission of radiation energy from the DNA fragment equipped with a radioactive label. The spacer sheet is preferably made of non or less radiation-transmitting material. Examples of the non or less radiation-transmitting materials include metals such as aluminum, brass and stainless and polymers such as polyethylene terephthalate and polyethylene naphthalate. The spacer sheet preferably has a thickness in the range of 10 to 300 μm.

In FIG. 3, the radiation image storage panel 19 is composed of a support sheet and a discontinuous stimulable phosphor layer 18, and has no protective film.

The stimulable phosphor layer generally comprises a stimulable phosphor in the form of particles and a binder resin.

A number of stimulable phosphors are already known and most of which are employable for the invention. Preferred are an alkaline earth metal halide activated by europium or cerium such as BaFBr:Eu and BaF(Br, I):Eu. A cerium activated rare earth oxyhalide phosphor is also preferred.

The stimulable phosphor layer can be formed on a support sheet by a known method.

The support sheet can preferably is a transparent or light-reflecting plastic material sheet or film. Examples of the plastic materials include polyethylene terephthalate, polyethylene naphthalate, polyamide, polyimide, and aramides resin. The thickness of the support sheet generally is in the range of 50 to 1,000 μm.

The stimulable phosphor layer can be formed, for example, in the following manner which is as such known.

First, the stimulable phosphor particles and a binder are placed in a solvent, and mixed well to prepare a coating liquid in which the phosphor particles are uniformly dispersed in a binder solution. As the binder, various resin materials are known and optionally usable for the invention. The ratio between the binder and the phosphor in the liquid depends on the characteristics of the phosphor and the aimed property of the phosphor layer, but generally they are employed at a ratio of 1:1 to 1:100 (binder:phosphor, by weight). The coating liquid may further contain various additives such as a dispersing agent (for promoting dispersing of the phosphor particles), a plasticizer (for improving binding between the phosphor particles and the binder), an anti-yellowing agent (for inhibiting yellowing of the phosphor layer), a hardening agent and a crosslinking agent.

The coating liquid thus prepared is evenly coated on a support (e.g., glass plate, metal plate, plastic sheet) by known coating means (such as doctor blade, roll coater, and knife coater), and dried to form a phosphor layer. The phosphor layer is once formed on a temporary sheet and then transferred onto the genuine support.

The stimulable phosphor layer can be a deposited phosphor layer or a sintered phosphor layer.

The process for detecting a complementary DNA fragment according to the invention is described below in more detail.

In the first step, single-stranded sample DNA fragments having a radioactive label is brought into contact with a DNA micro-array having two or more defined areas in each of which a group of probe compounds (nucleotide derivatives or their analogues such as DNA molecules, DNA fragments, synthesized oligonucleotides, synthesized polynucleotides, PNA) are fixed. A group of probe compounds fixed in one area differs from a group of probe compounds fixed in another area, so that DNA fragments complementary to a group of probe compounds are fixed by hybridization to the area in which the group is fixed. The single-stranded sample DNA fragments are generally supplied as a solution or dispersion in an aqueous medium.

Subsequently, unfixed sample DE fragments are removed from the DNA micro-array, for instance, by washing the surface of the DNA micro-array with an aqueous medium, so as to reduce noises and to improve accuracy of the analysis. The problem resides in the fact that DNA fragments which are not complementary to the probe compounds fixed onto the DNA micro-array are irregularly fixed to the surface of the micro-array, because the surface of the DNA micro-array sometimes has a great number of functional groups such as hydroxyl groups and amino groups and the DNA fragments also have various functional groups some of which are able to produce bonding with the functional groups on the micro-array.

The DNA micro-array having the sample DNA fragments on its surface is then subjected to autoradiography utilizing a radiation image storage panel. In the autoradiography, the DNA micro-array is kept in contact with a radiation image storage panel according to the invention.

The autoradiography is generally performed at a temperature in the range of 0 to 30° C., for one hour to 120 hours.

Generally, the radiation image storage panel is separated from the DNA micro-array.

The radiation image storage panel is then subjected to a known radiation image reproducing procedure. In the procedure, the radiation image storage panel is irradiated with a stimulating light, so that the image storage panel releases a stimulated emission from the area in which the radiation energy is stored. The stimulated emission is detected photoelectrically to obtain a series of electric signals. Finally, the electric signals are processed to locate the area in which the complementary DNA fragments are fixed.

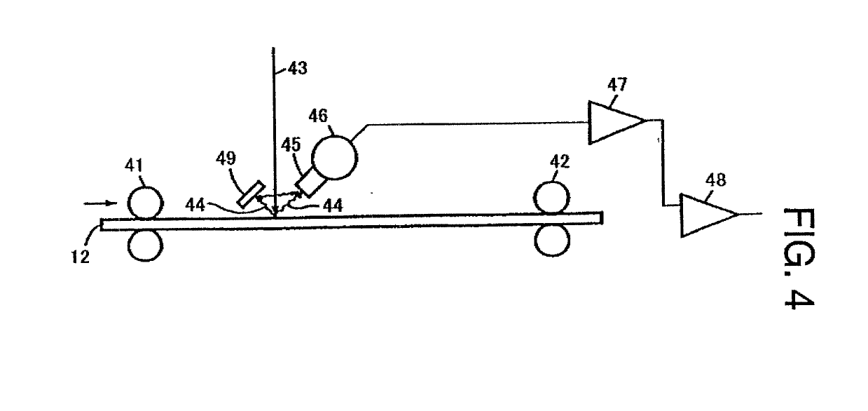

The typical radiation image reproducing procedure is illustrated in FIG. 4.

In FIG. 4, a radiation image storage panel 12 is transferred in the direction of arrow, by means of a pair of rollers 41. On the storage panel 12 is applied a stimulating light 43. A stimulated emission 44 is directly detected by a light detecting means 45 or indirectly detected after reflection on a mirror 49. In the photoelectric conversion means 46, the stimulated emission 44 is converted into a series of electric signals, which are then transmitted to a multiplier 47 and further processed in a processor 48.

What is claimed is:

1. A process for detecting a complementary DNA fragment which comprises the steps of:
bringing single-stranded sample DNA fragments having a radioactive label in a liquid phase into contact with a DNA micro-array having at least two defined areas in each of which a group of nucleotide derivatives and analogues thereof are fixed under such condition that a group of nucleotide derivatives and analogues thereof fixed in one area differs from a group of nucleotide derivatives and analogues thereof fixed in another area, so that DNA fragments complementary to a group of nucleotide derivatives and analogues thereof are fixed by hybridization to the area in which the group is fixed;
removing unfixed sample DNA fragments from the DNA micro-array;
keeping the DNA micro-array in contact with a radiation image storage panel containing a stimulable phosphor in areas corresponding to the areas on which groups of nucleotide derivatives or analogues thereof are fixed, so that the corresponding areas of the stimulable phosphor sheet can absorb and store radiation energy of the radioactive label coming from the fixed DNA fragments through the openings;
irradiating the radiation image storage panel with a stimulating light, so that the image storage panel releases a stimulated emission from the area in which the radiation energy is stored;
detecting the stimulated emission photoelectrically to obtain a series of electric signals; and
processing the electric signals to locate the area in which the complementary DNA fragments are fixed.

2. The process of claim 1, in which area on the radiation image storage panel other than the area of stimulable phosphor is covered by a barrier member.

3. The process of claim 1, in which the radiation image storage panel is irradiated with a stimulating light after it is separated from the DNA micro-array.

4. A kit for detecting complementary DNA fragments comprising a DNA micro-array having at least two defined areas in each of which a group of nucleotide derivatives and analogues thereof are fixed under such condition that a group of nucleotide derivatives and analogues thereof fixed in one area differs from a group of nucleotide derivatives and analogues thereof fixed in another area, and a radiation image storage panel containing a stimulable phosphor in areas corresponding to the areas on which groups of nucleotide derivatives or analogues thereof are fixed.

5. A composite structure comprising a DNA micro-array having at least two defined areas in each of which a group of nucleotide derivatives and analogues thereof are fixed under such condition that a group of nucleotide derivatives and analogues thereof fixed in one area differs from a group of nucleotide derivatives and analogues thereof fixed in another area, and a radiation image storage panel containing a stimulable phosphor in areas corresponding to the areas on which groups of nucleotide derivatives or analogues thereof are fixed, overlaid in order, the radiation image storage panel be positioned in relation to the DNA micro-array in such condition that the areas containing stimulable phosphor of the radiation image storage panel face the areas of the micro-array in which groups of nucleotide derivatives and analogues thereof are fixed.
